(19) United States
(12) Patent Application Publication
(10) Pub. No.: US 2006/0120975 A1
(43) Pub. Date: Jun. 8, 2006

(54) Oral care composition comprising a phenolic compound and antioxidant vitamins and vitamin derivatives

(75) Inventors: Dale Scherl, Lawrence, KS (US); Kimberlee Panaligan, Parlin, NJ (US); Linh Fruge, Hillsborough, NJ (US); Harsh M. Trivedi, Somerset, NJ (US); Tao Xu, East Brunswick, NJ (US)

Correspondence Address: COLGATE-PALMOLIVE COMPANY, 909 RIVER ROAD, PISCATAWAY, NJ, 08855, US

(73) Assignee: Colgate-Palmolive Company

(21) Appl. No.: 11/238,522

(22) Filed: Sep. 29, 2005

Related U.S. Application Data
(60) Provisional application No. 60/632,480, filed on Dec. 2, 2004.

(51) Int. Cl. A61K 8/49

(52) U.S. Cl. 424/49

(57) ABSTRACT

An oral care composition comprises an antibacterial phenolic compound and a vitamin or vitamin derivative component comprising one to a plurality of antioxidant vitamin or vitamin derivatives. In one embodiment the vitamin or vitamin derivative component comprises vitamin E and vitamin E acetate and optionally one or more additional vitamin or vitamin derivatives. In another embodiment the vitamin or vitamin derivative component comprises an antioxidant vitamin or vitamin derivative and a source of vitamin B₅. A method of oral care comprises applying a composition as described above to an oral surface of a subject.

### CROSS REFERENCE TO RELATED APPLICATION

This application claims the benefit of the prior filed U.S. Provisional Patent Application Ser. No. 60/632,480, filed 2 Dec. 2004, the contents of which are incorporated herein by reference.

### BACKGROUND OF THE INVENTION

Many phenolic compounds possess both antibacterial and anti-inflammatory properties, and are known to be useful as antibacterial and/or anti-inflammatory agents in oral care compositions such as mouthwashes and dentifrices. Examples of such phenolic compounds include carvacrol, eugenol (thoxyphenol), 4-hexylresorcinol (4-hexyl-1,3-dihydroxybenzene), bromochlorophene (2,2′-methylene bis(4-chloro-6-bromophenol)), thymol (2-isopropyl-5-methylphenol) and triclosan (5-chloro-2-(2,4-dichlorophenoxy)phenol).

Additionally, antioxidants have been proposed as ingredients of oral care compositions, for example as stabilizing agents operating by protecting readily oxidizable ingredients from oxidative degradation, and/or as agents to enhance oral and/or whole body health. Compounds disclosed as components of oral care compositions and described as antioxidants have included ascorbic acid (vitamin C), β-carotene, folic acid, α-lipoic acid (thioctic acid), lycopene, lutein (xanthophyll), niacin (nicotinamide and/or nicotinic acid), retinol (vitamin A), riboflavin (vitamin B₂), rutin, α-tocopherol or vitamin E, 6-hydroxy-2,5,7,8-tetramethylchroman-2-carboxylic acid, and ubiquinone (coenzyme Q₁₀). Such antioxidants are not reported to have any effect on the anti-inflammatory properties of dentifrices containing phenolic compounds.

### BRIEF SUMMARY OF THE INVENTION

There is now provided an oral care composition comprising at least one antibacterial halogenated diphenylether compound and an antioxidant component; wherein (a) the at least one halogenated diphenylether compound is present in an antibacterially effective total amount; (b) the antioxidant component comprises one to a plurality of vitamin or vitamin derivatives in a total vitamin or vitamin derivative amount effective to enhance the anti-inflammatory activity of the composition; and (c) at least one vitamin or vitamin derivative in the antioxidant component is ascorbic acid or an orally acceptable salt or ester thereof, or a 2-methyl-6-chromanol compound.

### DETAILED DESCRIPTION OF THE INVENTION

An “antibacterial phenolic compound” herein is a compound having a phenol moiety as part of its molecular structure, and exhibiting antibacterial activity when present in an oral care composition in an orally acceptable amount.

A “vitamin or vitamin derivative” herein is a compound that is a vitamin and/or has vitamin-like activity, including natural and synthetic vitamins as well as vitamin analogs, derivatives, precursors, esters, salts, isomers, racemates, enantiomers, tautomers and the like. A “source” of a vitamin herein can be the vitamin itself, a vitamin or vitamin derivative that upon administration to an oral surface generates or releases the vitamin on the oral surface and/or in an underlying tissue, or that otherwise exhibits vitamin activity characteristic of the vitamin. A “multivitamin or vitamin derivative complex” herein means a plurality of vitamin or vitamin derivatives.

Except where the context demands otherwise, the term “vitamin E” is used generically herein to encompass any tocopherol or tocotriene compound, including any enantiomer or racemate thereof, and any mixture of such compounds, having vitamin E activity.

When a “total” amount in a composition of “at least one” component that is a member of a specified class (e.g., the class of antibacterial phenolic compounds or the class of antioxidant vitamin or vitamin derivatives) is recited herein, it will be understood that the amount referred to is the total of the individual amounts of all members of the class present in the composition. Concentrations of ingredients herein are expressed by weight except as may otherwise be indicated.

A step of “applying” a composition to an oral surface herein encompasses any procedure that results in the composition contacting the surface, including irrigating, rinsing, spraying, wiping, rubbing, brushing, painting, flossing, placement of a film or strip on the surface, implanting and chewing.

The term “inhibiting” herein with respect to a condition such as inflammation in an oral tissue encompasses prevention, suppression, reduction in extent or severity, or amelioration of the condition.

An oral care composition of the present invention can take any physical form suitable for application to an oral surface. In various illustrative embodiments the composition can be a liquid solution suitable for irrigating, rinsing or spraying; a dentifrice such as a powder, toothpaste or dental gel; a periodontal gel; a liquid suitable for painting a dental surface (e.g., a liquid whitener); a chewing gum; a dissolvable, partially dissolvable or non-dissolvable film or strip (e.g., a whitening strip); a wafer; a wipe or towelette; an implant; a dental floss; etc. The composition can contain active and/or carrier ingredients additional to those recited above.

In certain embodiments the composition is adapted for application to an oral surface of a small domestic animal, for example a cat or a dog. Such a composition is typically edible or chewable by the animal, and can take the form, for example, of a cat or dog food, treat or toy.

The present invention is in part derived from a finding that, upon addition of an antioxidant vitamin or vitamin derivative to an antibacterial phenolic compound having anti-inflammatory activity, in particular a halogenated diphenylether compound having such activity, the anti-inflammatory activity can be enhanced. For example, addition of antioxidant vitamin or vitamin derivatives to the antibacterial phenolic compound triclosan has been found to lower to a surprising degree the IC₅₀ of triclosan in an IL-1β stimulated PGE2 production cell culture assay, an inflammation model. In this assay, ascorbic acid, vitamin E acetate and TROLOX® (a synthetic analogue of α-tocopherol) were found to be especially effective in enhancing the anti-inflammatory activity of triclosan. Ascorbyl palmitate and vitamin E exhibited less dramatic enhancement; it is believed without being bound by theory that solubility limitations may have reduced their efficacy in this assay.

Accordingly, in one embodiment of the invention, there is provided an oral care composition comprising at least one antibacterial halogenated diphenylether compound and an antioxidant component. In a composition of this embodiment the at least one halogenated diphenylether compound is present in an antibacterially effective total amount, and the antioxidant component comprises one to a plurality of vitamin or vitamin derivatives in a total vitamin or vitamin derivative amount effective to enhance the anti-inflammatory activity of the composition. Based on the finding summarized above, at least one vitamin or vitamin derivative in the antioxidant component should be ascorbic acid or an orally acceptable salt or ester thereof, or a 2-methyl-6-chromanol compound (for example vitamin E, vitamin E acetate or TROLOX®).

The at least one antibacterial halogenated diphenylether can be, for example, triclosan, triclosan monophosphate or 2,2′-dihydroxy-5,5′-dibromodiphenylether.

According to the present embodiment, the at least one halogenated diphenylether compound, e.g., triclosan, is typically present in the composition in a total amount of about 0.01% to about 10% by weight. The concentration depends in part on the form of the composition; for example, in a mouthwash or oral rinse a relatively low concentration, for example about 0.01% to about 0.5%, can be useful, whereas in a toothpaste or gel dentifrice a somewhat higher concentration, for example about 0.05% to about 5%, will generally be found satisfactory. Illustratively the total concentration of the at least one halogenated diphenylether compound in a toothpaste or gel dentifrice can be about 0.1% to about 2%, for example about 0.2% to about 1% or about 0.25% to about 0.5%.

Vitamins and vitamin derivatives useful herein can illustratively be selected from the following classes: (a) sources of vitamin C, including ascorbic acid; (b) 2-methyl-6-chromanol compounds, including TROLOX®, tocol (2-methyl-2-(4,8,12-trimethyltridecyl)-6-chromanol), α-tocopherol ((+)-2,5,7,8-tetramethyl-2-(4,8,12-trimethyltridecyl)-6-chromanol), β-tocopherol ((+)-2,5,8-trimethyl-2-(4,8,12-trimethyltridecyl)-6-chromanol), γ-tocopherol ((+)-2,7,8-trimethyl-2-(4,8,12-trimethyltridecyl)-6-chromanol), δ-tocopherol ((+)-8-methyl-2-(4,8,12-trimethyltridecyl)-6-chromanol), α-tocotrienol (2,5,7,8-tetramethyl-2-(4,8,12-trimethyl-3,7,11-tridecatrienyl)-6-chromanol), β-tocotrienol (2,5,8-trimethyl-2-(4,8,12-trimethyl-3,7,11-tridecatrienyl)-6-chromanol) and vitamin E (any one or a mixture of any two or more tocopherols and/or tocotrienols); (c) carotenoids, including retinol (vitamin A), retinal, retinoic acid, α-carotene, β-carotene, γ-carotene, δ-carotene, lutein, lycopene, lycophyll, lycoxanthin, rhodoxanthin, astaxanthin and cryptoxanthin; (d) sources of B vitamins, including thiamine (vitamin B₁), riboflavin (vitamin B₂), nicotinamide and nicotinic acid (both referred to as niacin), pantothenic acid (vitamin B₅), pantothenol, pyridoxine (vitamin B₆), pyridoxal, pyridoxamine, folic acid, dihydrofolic acid, vitamin B₁₂ and biotin; (e) bioflavonoids, including rutin, hesperetin, hesperidin, eriodictyol, quercetin, quercetagetin and quercetagitrin; (f) quinone-type enzyme cofactors, including ubiquinone (coenzyme Q₁₀) and pyrroloquinoline quinone (PQQ); (g) sources of α-lipoic acid; (h) sources of vitamin D, including calciferol and cholecalciferol; and (i) orally acceptable salts, esters (including phosphate, acetate and long-chain, e.g., linoleate and palmitate, esters), isomers, enantiomers, racemates and tautomers of the above.

Vitamins and vitamin derivatives useful herein can be natural or synthetic in origin and can be used in refined form or in crude form, for example as herbal preparations.

According to one aspect of the present embodiment, the antioxidant component comprises ascorbic acid and/or an orally acceptable salt or ester thereof. Illustrative salts include the sodium, calcium and zinc salts of ascorbic acid. An illustrative ester is ascorbyl palmitate. According to another aspect of the present embodiment, the antioxidant component comprises a 2-methyl-6-chromanol compound. Illustratively, the 2-methyl-6-chromanol compound can be TROLOX® or an orally acceptable salt thereof, or vitamin E or an orally acceptable ester thereof, for example vitamin E acetate.

According to yet another aspect of the present embodiment, the antioxidant component comprises (a) ascorbic acid and/or an orally acceptable salt or ester thereof, and (b) a 2-methyl-6-chromanol compound, for example as illustrated above.

The antioxidant component optionally further comprises one or more vitamin or vitamin derivatives other than a vitamin C source or a 2-methyl-6-chromanol compound. Such a vitamin or vitamin derivative can be, for example, a source of a B vitamin such as thiamine, riboflavin, niacin, vitamin B₅, vitamin B₆ or folic acid, a carotenoid such as β-carotene, lutein or lycopene, a bioflavonoid, a quinone-type enzyme cofactor or a source of α-lipoic acid.

The antioxidant component in a composition of the present embodiment is typically included in an amount providing a weight ratio of total antibacterial halogenated diphenylether compound(s) to total vitamin or vitamin derivative(s) of about 10:1 to about 1:100, for example about 5:1 to about 1:50, or about 2:1 to about 1:20, or about 1:1 to about 1:10.

Enhancement of anti-inflammatory activity is not the only advantage that can be gained by providing an oral care composition having one or more antibacterial phenolic compounds and one or more antioxidant vitamin or vitamin derivatives. For example, concurrent application of an antibacterial agent and an antioxidant to an oral surface can provide complementary benefits in oral health, especially in the periodontal area. Furthermore, the vitamin or vitamin-like activity of the vitamin or vitamin derivative component can provide oral and systemic health benefits above and beyond its antioxidant effect. In the case of vitamin E, it has been found desirable to include both an esterified and a non-esterified form of the vitamin. Without being bound by theory, it is believed that having both esterified and non-esterified forms enhances delivery and hence efficacy of the vitamin as an antioxidant. For example, vitamin E can give a short-burst effect while vitamin E acetate provides longer-term benefit.

Accordingly, in another embodiment of the invention, there is provided an oral care composition comprising at least one antibacterial phenolic compound in an antibacterially effective amount, and a vitamin or vitamin derivative component that comprises vitamin E and vitamin E acetate. In a composition of this embodiment, the at least one phenolic compound and the vitamin or vitamin derivative component are present in a total weight ratio of about 10:1 to about 1:100.

Phenolic compounds useful herein illustratively include, subject to determination of oral acceptability, those identified as having anti-inflammatory activity by Dewhirst (1980), Prostaglandins 20(2), 209-222, but are not limited thereto. Examples of antibacterial phenolic compounds include 4-allylcatechol, p-hydroxybenzoic acid esters including benzylparaben, butylparaben, ethylparaben, methylparaben and propylparaben, 2-benzylphenol, butylated hydroxyanisole, butylated hydroxytoluene, capsaicin, carvacrol, creosol, eugenol, guaiacol, halogenated bisphenolics including hexachlorophene and bromochlorophene, 4-hexylresorcinol, 8-hydroxyquinoline and salts thereof, salicylic acid esters including menthyl salicylate, methyl salicylate and phenyl salicylate, phenol, pyrocatechol, salicylanilide, thymol, triclosan and triclosan monophosphate.

The at least one antibacterial phenolic compound is in one aspect a halogenated diphenylether, for example triclosan, triclosan monophosphate or 2,2′-dihydroxy-5,5′-dibromodiphenylether.

The at least one phenolic compound is present in a total amount of about 0.01% to about 10% by weight. Illustratively the total concentration of the at least one phenolic compound in a toothpaste or gel dentifrice of the present embodiment can be about 0.05% to about 5%, for example about 0.1% to about 2%, about 0.2% to about 1% or about 0.25% to about 0.5%.

The vitamin or vitamin derivative component comprises vitamin E, for example in the form of α-tocopherol, and vitamin E acetate, for example in the form of α-tocopheryl acetate. The vitamin E and vitamin E acetate fractions can be in any suitable weight ratio, for example about 20:1 to about 1:10, or about 15:1 to about 1:5, or about 5:1 to about 1:1. Illustratively the total concentration of vitamin E and vitamin E acetate in a toothpaste or gel dentifrice can be about 0.05% to about 5%, for example about 0.1% to about 2.5% or about 0.2% to about 1%.

According to one aspect of the present embodiment, the vitamin or vitamin derivative component can further comprise one or more vitamin or vitamin derivatives other than vitamin E and vitamin E acetate. As one example, the composition optionally further comprises at least one source of vitamin C such as ascorbic acid, sodium ascorbate, calcium ascorbate, zinc ascorbate or ascorbyl palmitate. As another example, the composition optionally further comprises at least one carotenoid such as retinol, retinoic acid or an orally acceptable salt thereof, vitamin A palmitate, β-carotene, lutein or lycopene. As yet another example, the composition optionally further comprises at least one source of a B vitamin such as thiamine, riboflavin, niacin, vitamin B₅, vitamin B₆ or folic acid. As yet another example, the composition optionally further comprises at least one bioflavonoid such as rutin or a derivative thereof, for example rutin sulfuric acid ester, sodium salt. As yet another example, the composition optionally further comprises at least one quinone-type enzyme cofactor such as ubiquinone or PQQ. As yet another example, the composition further comprises at least one source of α-lipoic acid.

According to another aspect of the present embodiment, the vitamin or vitamin derivative component can be a multivitamin or vitamin derivative complex comprising, in addition to vitamin E and vitamin E acetate, a plurality of vitamin or vitamin derivatives selected from at least two of the following classes: (a) sources of vitamin C; (b) carotenoids; (c) sources of B vitamins; (d) bioflavonoids; (e) quinone-type enzyme cofactors; (f) sources of α-lipoic acid; and (g) sources of vitamin D.

Illustratively and without limitation, the following examples of a multivitamin or vitamin derivative complex, wherein any vitamin listed in acid form can optionally be wholly or partly in the form of an orally-acceptable salt or ester, can be useful according to the present embodiment:
1. vitamin E+vitamin E acetate;
2. vitamin E+vitamin E acetate+ascorbic acid;
3. vitamin E+vitamin E acetate+ascorbyl palmitate;
4. vitamin E+vitamin E acetate+β-carotene;
5. vitamin E+vitamin E acetate+lycopene;
6. vitamin E+vitamin E acetate+ascorbic acid+β-carotene;
7. vitamin E+vitamin E acetate+ascorbic acid+β-carotene+lycopene;
8. vitamin E+vitamin E acetate+pantothenic acid;
9. vitamin E+vitamin E acetate+ascorbic acid+pantothenic acid;
10. vitamin E+vitamin E acetate+vitamin B₆;
11. vitamin E+vitamin E acetate+ascorbic acid+vitamin B₆;
12. vitamin E+vitamin E acetate+ascorbic acid+pantothenic acid+vitamin B₆;
13. vitamin E+vitamin E acetate+ascorbic acid+β-carotene+lycopene+pantothenic acid+vitamin B₆+folic acid;
14. vitamin E+vitamin E acetate+rutin;
15. vitamin E+vitamin E acetate+ubiquinone;
16. vitamin E+vitamin E acetate+α-lipoic acid;
17. vitamin E+vitamin E acetate+calciferol;
18. vitamin E+vitamin E acetate+ascorbic acid+β-carotene+lycopene+pantothenic acid+vitamin B₆+folic acid+rutin+ubiquinone+α-lipoic acid.

The vitamin or vitamin derivative component in a composition of the present embodiment is illustratively included in an amount providing a weight ratio of total antibacterial phenolic compound to total vitamin or vitamin derivative component of about 5:1 to about 1:50, for example about 2:1 to about 1:20, or about 1:1 to about 1:10.

In yet another embodiment of the invention, there is provided an oral care composition comprising at least one antibacterial halogenated diphenylether compound in an antibacterially effective amount, and a vitamin or vitamin derivative component that comprises (a) one or more antioxidant vitamin or vitamin derivatives selected from the group consisting of ascorbic acid, orally acceptable salts and esters thereof, and 2-methyl-6-chromanol compounds, and (b) at least one source of vitamin B₅. It will be understood that, in the context of this embodiment, the “one or more antioxidant vitamin or vitamin derivatives” are other than a source of vitamin B₅ and are additional thereto. The source of vitamin B₅ may or may not function as an antioxidant in the composition. In a composition of this embodiment, the at least one halogenated diphenylether compound and the vitamin or vitamin derivative component are present in a total weight ratio of about 10:1 to about 1:100.

The at least one antibacterial halogenated diphenylether compound can suitably be selected from triclosan, triclosan monophosphate and 2,2′-dihydroxy-5,5′-dibromodiphenylether.

The at least one halogenated diphenylether compound is again present in a total amount of about 0.01% to about 10% by weight. Illustratively the total concentration of the at least one halogenated diphenylether compound in a toothpaste or gel dentifrice can be about 0.05% to about 5%, for example about 0.1% to about 2%, about 0.2% to about 1% or about 0.25% to about 0.5%.

In one aspect of the present embodiment, the vitamin or vitamin derivative component comprises a natural or synthetic source of vitamin E, for example α-tocopherol or vitamin E acetate, e.g., as α-tocopheryl acetate. Optionally both vitamin E and vitamin E acetate are present, for example in a weight ratio of about 20:1 to about 1:10, or about 15:1 to about 1:5, or about 5:1 to about 1:1. Illustratively the total concentration of antioxidant vitamin or vitamin derivatives other than sources of vitamin B₅, for example sources of vitamin E, in a toothpaste or gel dentifrice can be about 0.05% to about 5%, for example about 0.1% to about 2.5% or about 0.2% to about 1%.

The vitamin or vitamin derivative component according to the present embodiment comprises at least one natural or synthetic source of vitamin B₅, for example pantothenic acid or an orally acceptable salt or ester thereof, or pantothenol. The at least one source of vitamin B₅ can illustratively be present in a total amount such that the weight ratio of sources of vitamin B₅ to antioxidant vitamin or vitamin derivatives other than sources of vitamin B₅ is about 1:100 to about 1:1, for example about 1:50 to about 1:5. The total concentration of sources of vitamin B₅ in a toothpaste or gel dentifrice can illustratively be about 0.005% to about 1%, for example about 0.01% to about 0.5% or about 0.02% to about 0.2%.

According to another aspect of the present embodiment, the vitamin or vitamin derivative component can be a multivitamin or vitamin derivative complex comprising a source of vitamin B₅ (e.g., pantothenic acid or an orally acceptable salt or ester thereof) and a plurality of vitamin or vitamin derivatives selected from at least two of the following classes: (a) sources of vitamin E; (b) sources of vitamin C; (c) carotenoids; (d) sources of B vitamins other than vitamin B₅; (e) bioflavonoids; (f) quinone-type enzyme cofactors; (g) sources of α-lipoic acid; and (h) sources of vitamin D; wherein at least one of the plurality of vitamin or vitamin derivatives is an antioxidant.

Illustratively and without limitation, the following examples of a multivitamin or vitamin derivative complex, wherein any vitamin listed in acid form can optionally be wholly or partly in the form of an orally-acceptable salt or ester, can be useful according to the present embodiment:
1. vitamin E+pantothenic acid;
2. vitamin E+ascorbic acid+pantothenic acid;
3. ascorbic acid+pantothenic acid;
4. vitamin E+pantothenic acid+β-carotene;
5. vitamin E+pantothenic acid+lycopene;
6. vitamin E+ascorbic acid+pantothenic acid+β-carotene;
7. vitamin E+ascorbic acid+pantothenic acid+β-carotene+lycopene;
8. vitamin E+pantothenic acid+vitamin B₆;
9. ascorbic acid+pantothenic acid+vitamin B₆;
10. vitamin E+ascorbic acid+pantothenic acid+vitamin B₆;
11. vitamin E+ascorbic acid+pantothenic acid+β-carotene+lycopene+vitamin B₆+folic acid;
12. vitamin E+pantothenic acid+rutin;
13. vitamin E+pantothenic acid+ubiquinone;
14. vitamin E+pantothenic acid+α-lipoic acid;
15. vitamin E+pantothenic acid+calciferol;
16. vitamin E+ascorbic acid+pantothenic acid+β-carotene+lycopene+vitamin B₆+folic acid+rutin+ubiquinone+α-lipoic acid.

Pantothenol (also known as panthenol, for example DL-panthenol) can optionally be substituted in whole or in part for pantothenic acid or a salt or ester thereof in any of the above illustrative multivitamin or vitamin derivative complexes.

The vitamin or vitamin derivative component in a composition of the present embodiment is illustratively included in an amount providing a weight ratio of total antibacterial halogenated diphenylether compound to total vitamin or vitamin derivative component of about 5:1 to about 1:50, for example about 2:1 to about 1:20, or about 1:1 to about 1:10.

Illustratively, the composition of any of the embodiments described above is a mouthwash or rinse, an oral spray, a dentifrice, an oral strip, a liquid whitener or a chewing gum. Rinses include liquids adapted for irrigation by means of devices such as high-pressure water jets. Dentifrices include without limitation toothpastes, gels and powders. A “liquid whitener” herein encompasses semi-liquid compositions such as gels as well as flowable liquids, so long as the composition is capable of application to a dental surface by painting with a brush or other suitable device. “Painting” in the present context means application of a thin layer of the composition to the dental surface. In one embodiment the composition is a toothpaste or gel dentifrice.

A composition of the invention can comprise, in addition to the at least one antibacterial phenolic (for example halogenated diphenylether) compound and a vitamin or vitamin derivative or antioxidant component as defined above, one or more active agents (“actives”).

Among useful actives are those addressing, without limitation, appearance and structural changes to teeth, treatment and prevention of plaque, calculus, dental caries, cavities, abscesses, inflamed and/or bleeding gums, gingivitis, oral infective and/or inflammatory conditions in general, tooth sensitivity, halitosis and the like. Thus, a composition of the invention can contain one or more actives such as whitening agents, fluoride ion sources, antimicrobial agents additional to the at least one antibacterial phenolic (for example halogenated diphenylether) compound, desensitizing agents, anticalculus (tartar control) agents, stannous ion sources, zinc ion sources, sialagogues, breath-freshening agents, antiplaque agents, anti-inflammatory agents additional to any anti-inflammatory phenolic compound present, periodontal agents, analgesics and nutrients additional to the at least one vitamin or vitamin derivative. Actives should be selected for compatibility with each other and with other ingredients of the composition.

Actives useful herein are normally present in the composition in amounts selected to be safe and effective, i.e., amount sufficient to provide a desired benefit, for example a therapeutic, prophylactic, nutritive or cosmetic effect, when the composition is used repeatedly as described herein, without undue side effects such as toxicity, irritation or allergic reaction, commensurate with a reasonable benefit/risk ratio. Such a safe and effective amount will usually, but not necessarily, fall within ranges approved by appropriate regulatory agencies. A safe and effective amount in a specific case depends on many factors, including the particular benefit desired or condition being treated or sought to be prevented, the particular subject using, or being administered, the composition, the frequency and duration of use, etc. Actives are typically present in a total amount of about 0.01% to about 80%, for example about 0.05% to about 60%, about 0.1% to about 50%, or about 0.5% to about 40%, by weight of the composition.

One or more actives, including the antibacterial agent and/or the vitamin or vitamin derivative component herein, can optionally be present in encapsulated form in the composition. For example, beads containing one or more actives can be adapted to rupture during brushing or chewing to release the active(s) to the oral surface.

Additionally, the composition of the invention may include any of the components conventionally present or desirable in an oral care product. For example, the composition may include a whitening agent, such as peroxy compounds, chlorine dioxide, chlorites and hypochlorites, a polymer-peroxide complex, polyvinylpyrrolidone-hydrogen peroxide (PVP-H₂O₂) complex; a source of fluride ions (monofluorophosphate and fluorosilicate salts, antibacterial agents. Active agents such as antibacterial agents may be includes, including, for example, those listed in U.S. Pat. No. 5,776,435 to Gaffar et al., the contents of which are incorporated herein by reference. The composition may further include a tooth anti-sensitivity agent, a sialagogue (saliva stimulating agent), a breath-freshening agent, an antiplaque or plaque disrupting agent.

Among useful carriers for optional inclusion in a composition of the invention are diluents, abrasives, bicarbonate salts, pH modifying agents, surfactants, foam modulators, thickening agents, viscosity modifiers, humectants, sweeteners, flavorants and colorants. One carrier material, or more than one carrier material of the same or different classes, can optionally be present. Water is a preferred diluent and in some compositions such as mouthwashes and whitening liquids may be accompanied by an additional solvent, such as an alcohol, e.g., ethanol.

The composition may contain abrasives, pH modifying agents, surfactants, foam modulators, thickening agents, viscosity modifiers, humectants, sweeteners, flavorants, colorants.

The invention further provides a method of oral care comprising a step of applying a composition as described herein to an oral surface of a subject. In one embodiment the composition is a toothpaste or gel dentifrice, and the applying step comprises brushing the surface, for example a dental surface and a periodontal surface adjacent thereto, with the dentifrice.

According to an embodiment of the invention, there is still further provided a method of inhibiting inflammation in an oral tissue of a subject. The method of this embodiment comprises applying to an oral surface proximal to the tissue a composition of the inventions. In another embodiment, there is provided a method of promoting oral health in a subject. The method of this embodiment comprises applying to an oral surface of the subject a composition of the inventions.

Practice of a method of the invention can promote any aspect or aspects of oral health. As one example, such a method can promote periodontal and/or gingival health, for instance by reducing bacterial infection and/or inflammation. As another example, such a method can provide a breath-freshening benefit, for instance through antioxidant activity. As yet another example, such a method can promote tooth retention, for instance by reducing or preventing dental caries and preventing destruction of the bone matrix that holds the tooth in place. As yet another example, such a method can provide an anti-plaque benefit. As yet another example, such a method can aid the subject's defense mechanisms against oral cancers and precancerous conditions. As yet another example, such a method can reduce damage to oral tissues from free radicals, including those occurring as a result of contact with tobacco smoke or polluted air.

It is well known that enhanced oral health, in particular improved periodontal and/or gingival health associated with reduced bacterial infection and/or inflammation, can lead to systemic or whole-body health benefits. Delivery of vitamins via an oral surface as provided herein can further enhance general health by supplementing the vitamins ingested with food.

Among systemic conditions that can be ameliorated as a result of improved oral health following practice of a method of the invention are cardiovascular disease including atherosclerosis, coronary heart disease (CHD) and stroke; diabetes; respiratory infections including bacterial pneumonia; preterm low birth weight; stomach ulcers; bacteremia; infective endocarditis; prosthetic device infection; chronic obstructive pulmonary disease (COPD); behavior and psychosocial disorders; and brain abscesses.

Practice of the methods can consist of a single application as described herein, or can comprise repeated such applications. In one embodiment a method as described herein is repeated at regular intervals, for example twice or once daily, twice or once weekly, twice or once monthly, in a program or regimen conducted at home and/or in a professional or clinical setting.

The subject in any of the above methods can be a human or non-human mammal, for example a dog, cat, horse or exotic mammal. In certain embodiments the subject is a small domestic animal, for example a cat or a dog, and the composition, in the form of a food, treat or toy, is given to the animal to chew.

The invention can further be understood by reference to the following nonlimiting examples.

### EXAMPLES

### Example 1

Toothpaste compositions were prepared having ingredients as shown in Table 1. Comparative composition 1Z was a conventionally prepared glycerin/sorbitol-based toothpaste containing silica abrasives, a PVME/MA (GANTREZ®) anticalculus agent, sodium fluoride and other conventional ingredients. Triclosan was present at 0.3%. Composition 1A of the invention was substantially identical except for addition, at the final stage of mixing, of vitamin E acetate and DL-panthenol as indicated in Table 1.

TABLE 1
Composition of toothpastes (percent by weight)

Composition No.

| Ingredient | 1Z | 1A |
|---|---|---|
| toothpaste base¹ | 99.70 | 99.55 |
| triclosan | 0.30 | 0.30 |
| vitamin E acetate | | 0.10 |
| DL-panthenol | | 0.05 |

¹toothpaste base included the following ingredients: silica powder, glycerin, sorbitol, water, GANTREZ ®, sodium lauryl sulfate, sodium hydroxide, titanium dioxide, flavor, sodium CMC, ι-carrageenan, sodium fluoride, sodium saccharin.

### Example 2

Toothpaste compositions were prepared having ingredients as shown in Table 2. Comparative composition 2Z was a conventionally prepared sorbitol-based toothpaste containing silica abrasives, a PVME/MA (GANTREZ®) anticalculus agent, sodium fluoride and other conventional ingredients. Triclosan was present at 0.3%. Composition 2A of the invention was substantially identical except for addition, at the final stage of mixing, of vitamin E acetate and DL-panthenol as indicated in Table 2.

TABLE 2
Composition of toothpastes (percent by weight)

Composition No.

| Ingredient | 2Z | 2A |
|---|---|---|
| toothpaste base² | 99.70 | 99.55 |
| triclosan | 0.30 | 0.30 |
| vitamin E acetate | | 0.10 |
| DL-panthenol | | 0.05 |

²toothpaste base included the following ingredients: sorbitol, water, silica powder, GANTREZ ®, sodium lauryl sulfate, sodium hydroxide, titanium dioxide, flavor, ι-carrageenan, glycerin, sodium fluoride, sodium saccharin.

### Example 3

Toothpaste compositions were prepared having ingredients as shown in Table 3. Comparative composition 3Z was a conventionally prepared glycerin/sorbitol-based toothpaste containing silica abrasives, a PVME/MA (GANTREZ®) anticalculus agent, sodium fluoride and other conventional ingredients. Triclosan was present at 0.3%. Compositions 3A-3F of the invention were substantially identical except for addition, at the final stage of mixing, of vitamins as indicated in Table 3.

TABLE 3
Composition of toothpastes (percent by weight)

| | Composition No. | | | | | | |
|---|---|---|---|---|---|---|---|
| Ingredient | 3Z | 3A | 3B | 3C | 3D | 3E | 3F |
| toothpaste base³ | 99.70 | 97.40 | 98.40 | 99.15 | 99.30 | 98.45 | 99.10 |
| Triclosan | 0.30 | 0.30 | 0.30 | 0.30 | 0.30 | 0.30 | 0.30 |
| vitamin E acetate | | 0.20 | 0.20 | 0.20 | 0.20 | 0.20 | 0.20 |
| vitamin E | | 2.00 | 1.00 | 0.30 | 0.10 | 1.00 | 0.30 |
| DL-panthenol | | 0.05 | 0.05 | 0.05 | 0.05 | 0.05 | 0.05 |
| vitamin C | | 0.05 | 0.05 | | 0.05 | | 0.05 |

³toothpaste base included the following ingredients: water, silica powder, sorbitol, glycerin, GANTREZ ®, sodium lauryl sulfate, sodium hydroxide, sodium CMC, propylene glycol, titanium dioxide, sodium saccharin, sodium fluoride.

### Example 4

Toothpaste compositions were prepared having ingredients as shown in Table 4. Comparative composition 4Z was a conventionally prepared sorbitol-based toothpaste containing silica abrasives, a PVME/MA (GANTREZ®) anticalculus agent, sodium fluoride and other conventional ingredients. Triclosan was present at 0.3%. Composition 4A of the invention was substantially identical except for addition, at the final stage of mixing, of vitamin E acetate, vitamin E and DL-panthenol as indicated in Table 4.

TABLE 4
Composition of toothpaste (percent by weight)

Composition No.

| Ingredient | 4Z | 4A |
|---|---|---|
| toothpaste base⁴ | 99.70 | 99.55 |
| triclosan | 0.30 | 0.30 |
| vitamin E acetate | | 0.10 |
| vitamin E | | 0.30 |
| DL-panthenol | | 0.05 |

⁴toothpaste base included the following ingredients: sorbitol, water, silica powder, propylene glycol, GANTREZ ®, sodium lauryl sulfate, sodium hydroxide, ι-carrageenan, titanium dioxide, sodium saccharin, sodium fluoride.

We claim:

1. An oral care composition comprising an antibacterial halogenated diphenylether compound and an antioxidant component, wherein the antioxidant component comprises one or more of a vitamin or vitamin derivative; wherein at least one of the one or more vitamins or vitamin derivatives is ascorbic acid, its salt or ester, or 2-methyl-6-chromanol.

2. The composition of claim 1 wherein the halogenated diphenylether compound is triclosan.

3. The composition of claim 1 wherein the halogenated diphenylether compound is present in an amount of about 0.01% to about 10% by weight of the composition.

4. The composition of claim 1 in a form of a toothpaste or gel dentifrice.

5. The composition of claim 5 wherein the at least one halogenated diphenylether compound is present in a total amount of about 0.05% to about 5% by weight.

6. The composition of claim 1 wherein the antioxidant component comprises ascorbic acid and/or an orally acceptable salt or ester thereof.

7. The composition of claim 1 wherein the antioxidant component comprises at least one 2-methyl-6-chromanol compound.

8. The composition of claim 8 wherein the at least one 2-methyl-6-chromanol compound is selected from the group consisting of 6-hydroxy-2,5,7,8-tetramethylchroman-2-carboxylic acid and orally acceptable salts thereof, vitamin E and orally acceptable vitamin E esters.

9. The composition of claim 1 wherein the antioxidant component comprises ascorbic acid and/or an orally acceptable salt or ester thereof, and at least one 2-methyl-6-chromanol compound.

10. The composition of claim 10 wherein the at least one 2-methyl-6-chromanol compound is selected from the group consisting of 6-hydroxy-2,5,7,8-tetramethylchroman-2-carboxylic acid and orally acceptable salts thereof, vitamin E and orally acceptable vitamin E esters.

11. The composition of claim 1 wherein the antioxidant component further comprises at least one vitamin or vitamin derivative selected from the group consisting of sources of B vitamins, carotenoids, bioflavonoids, quinone-type enzyme cofactors, sources of α-lipoic acid, and mixtures thereof.

12. The composition of claim 1 wherein the at least one halogenated diphenylether compound and the vitamin or vitamin derivative(s) are present in a total weight ratio of about 10:1 to about 1:100.

13. The composition of claim 1 wherein the at least one halogenated diphenylether compound and the vitamin or vitamin derivative(s) are present in a total weight ratio of about 1:1 to about 1:10.

14. An oral care composition comprising at least one antibacterial phenolic compound in an antibacterially effective amount, and a vitamin or vitamin derivative component that comprises vitamin E and vitamin E acetate; wherein the at least one phenolic compound and the vitamin or vitamin derivative component are present in a total weight ratio of about 10:1 to about 1:100.

15. The composition of claim 15 in a form of a liquid solution suitable for irrigating, rinsing or spraying; a powder, toothpaste or gel dentifrice; a periodontal gel; a liquid suitable for painting a dental surface; a chewing gum; a dissolvable, partially dissolvable or non-dissolvable film or strip; a wafer; a wipe or towelette; an implant; or a dental floss.

16. The composition of claim 15 that is edible or chewable by a small domestic animal.

17. The composition of claim 15, wherein the at least one phenolic compound is selected from the group consisting of 4-allylcatechol, benzylparaben, 2-benzylphenol, bromochlorophene, butylated hydroxyanisole, butylated hydroxytoluene, butylparaben, capsaicin, carvacrol, creosol, ethylparaben, eugenol, guaiacol, hexachlorophene, 4-hexylresorcinol, 8-hydroxyquinoline and salts thereof, menthyl salicylate, methylparaben, methyl salicylate, phenol, phenyl salicylate, propylparaben, pyrocatechol, salicylanilide, thymol, triclosan, triclosan monophosphate and mixtures thereof.

18. The composition of claim 15, wherein the at least one phenolic compound is a halogenated diphenylether.

19. The composition of claim 15, wherein the at least one phenolic compound is triclosan.

20. The composition of claim 15, wherein the at least one phenolic compound is present in a total amount of about 0.01% to about 10% by weight.

21. The composition of claim 15, in a form of a toothpaste or gel dentifrice.

22. The composition of claim 22 wherein the at least one phenolic compound is present in a total amount of about 0.05% to about 5% by weight.

23. The composition of claim 23 wherein the vitamin E and vitamin E acetate are present in a total amount of about 0.05% to about 5% by weight.

24. The composition of claim 15 wherein the vitamin E is in the form of α-tocopherol, and the vitamin E acetate is in the form of α-tocopheryl acetate.

25. The composition of claim 15 wherein the vitamin E and vitamin E acetate are present in a weight ratio of about 10:1 to about 1:10.

26. The composition of claim 15 wherein the vitamin or vitamin derivative component further comprises at least one source of vitamin C.

27. The composition of claim 15 wherein the vitamin or vitamin derivative component further comprises at least one carotenoid.

28. The composition of claim 15 wherein the vitamin or vitamin derivative component further comprises at least one source of a B vitamin.

29. The composition of claim 15 wherein the vitamin or vitamin derivative component further comprises at least one bioflavonoid.

30. The composition of claim 15 wherein the vitamin or vitamin derivative component further comprises at least one quinone-type enzyme cofactor.

31. The composition of claim 15 wherein the vitamin or vitamin derivative component further comprises at least one source of α-lipoic acid.

32. The composition of claim 15 wherein the vitamin or vitamin derivative component is a multivitamin or vitamin derivative complex comprising vitamin E, vitamin E acetate, and a plurality of vitamin or vitamin derivatives selected from at least two of (a) sources of vitamin C; (b) carotenoids; (c) sources of B vitamins; (d) bioflavonoids; (e) quinone-type enzyme cofactors; (f) sources of α-lipoic acid; and (g) sources of vitamin D.

33. An oral care composition comprising at least one antibacterial halogenated diphenylether compound in an antibacterially effective amount, and a vitamin or vitamin derivative component that comprises (a) one or more antioxidant vitamin or vitamin derivatives selected from the group consisting of ascorbic acid, orally acceptable salts and esters thereof, and 2-methyl-6-chromanol compounds, and (b) at least one source of vitamin B₅; wherein the at least one halogenated diphenylether compound and the vitamin or vitamin derivative component are present in a total weight ratio of about 10:1 to about 1:100.

34. A method of oral care comprising applying to an oral surface of a subject a composition that comprises at least one antibacterial halogenated diphenylether compound and an antioxidant component; wherein (a) the at least one halogenated diphenylether compound is present in an antibacterially effective total amount; (b) the antioxidant component comprises one to a plurality of vitamin or vitamin derivatives in a total vitamin or vitamin derivative amount effective to enhance the anti-inflammatory activity of the composition; and (c) at least one vitamin or vitamin derivative in the antioxidant component is ascorbic acid or an orally acceptable salt or ester thereof, or a 2-methyl-6-chromanol compound.

35. A method of inhibiting inflammation in an oral tissue of a subject, the method comprising applying to an oral surface proximal to the tissue a composition that comprises at least one antibacterial halogenated diphenylether compound and an antioxidant component; wherein (a) the at least one halogenated diphenylether compound is present in an antibacterially effective total amount; (b) the antioxidant component comprises one to a plurality of vitamin or vitamin derivatives in a total vitamin or vitamin derivative amount effective to enhance the anti-inflammatory activity of the composition; and (c) at least one vitamin or vitamin derivative in the antioxidant component is ascorbic acid or an orally acceptable salt or ester thereof, or a 2-methyl-6-chromanol compound.

36. A method of promoting oral health in a subject, the method comprising applying to an oral surface of the subject a composition that comprises at least one antibacterial phenolic compound in an antibacterially effective amount, and a vitamin or vitamin derivative component that comprises vitamin E and vitamin E acetate; wherein the at least one phenolic compound and the vitamin or vitamin derivative component are present in a total weight ratio of about 10:1 to about 1:100.

37. A method of promoting oral health in a subject, the method comprising applying to an oral surface of the subject a composition that comprises at least one antibacterial halogenated diphenylether compound in an antibacterially effective amount, and a vitamin or vitamin derivative component that comprises (a) one or more antioxidant vitamin or vitamin derivatives selected from the group consisting of ascorbic acid, orally acceptable salts and esters thereof, and 2-methyl-6-chromanol compounds, and (b) at least one source of vitamin B₅; wherein the at least one halogenated diphenylether compound and the vitamin or vitamin derivative component are present in a total weight ratio of about 10:1 to about 1:100.
